(19) United States
(12) Patent Application Publication
(10) Pub. No.: US 2023/0145790 A1
(43) Pub. Date: May 11, 2023

(54) DEVICE, COMPUTER PROGRAM AND METHOD

(71) Applicant: Sony Group Corporation, Tokyo (JP)

(72) Inventors: Nicholas WALKER, London (GB); Christopher WRIGHT, London (GB); Akinori KAMODA, Tokyo (JP)

(73) Assignee: Sony Group Corporation, Tokyo (JP)

(21) Appl. No.: 17/917,583

(22) PCT Filed: Jun. 7, 2021
(86) PCT No.: PCT/JP2021/021579

§ 371 Date: Oct. 7, 2022

(30) Foreign Application Priority Data
Jul. 14, 2020 (EP) 20185660.6

(51) Int. Cl. A61B 34/30; A61B 34/00; A61B 90/00

(52) U.S. Cl. CPC A61B 34/30; A61B 34/25; A61B 90/361; A61B 34/10

(57) ABSTRACT

A device, comprising circuitry configured to: receive surgical data indicating a parameter of a surgical instrument used during a surgical procedure and a stress related parameter of a member of a surgical team performing the surgical procedure; and associating the surgical data with the stress related parameter.

### TECHNICAL FIELD

The present technique relates to a device, computer program and method.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present technique.

Stress levels for surgeons are very high during surgery. Whilst experienced surgeons manage these stress levels, trainee surgeons may suffer burnout during training. This is especially the case with surgical procedures having a slower learning rate such as endoscopy and minimally invasive surgery.

It is therefore desirable to manage stress levels of trainee surgeons during training whilst providing good quality training for the surgeon.

It is an aim of the disclosure to address this issue.

### SUMMARY

According to embodiments, there is provided a device, comprising circuitry configured to: receive surgical data indicating a parameter of a surgical instrument used during a surgical procedure and a stress related parameter of a member of a surgical team performing the surgical procedure; and associating the surgical data with the stress related parameter.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 shows surgery on a patient 106 by an experienced surgeon.

FIG. 2 shows some components of the control apparatus 100.

FIG. 3 shows a system 300 according to embodiments of the disclosure.

FIG. 4 shows the function of the medical procedure server 315 according to embodiments of the disclosure.

FIG. 5 shows a data structure according to embodiments of the disclosure.

FIG. 6 shows a data structure of a different surgical procedure carried out by a different expert surgeon according to embodiments of the disclosure.

FIG. 7 shows a second data structure according to embodiments of the disclosure.

FIG. 8 shows a training schedule for a simulation that manages the stress levels of the surgeon undergoing training according to embodiments.

FIG. 9A shows a training graph according to embodiments.

FIG. 9B shows a training graph according to embodiments.

FIG. 10 shows a system 1000 according to embodiments of the disclosure.

FIG. 11A shows embodiments of the 1^{st} part of the disclosure.

FIG. 11B shows embodiments of the 2^{nd} part of the disclosure.

FIG. 12 schematically shows a first example of a computer assisted surgery system to which the present technique is applicable.

FIG. 13 schematically shows a second example of a computer assisted surgery system to which the present technique is applicable.

FIG. 14 schematically shows a third example of a computer assisted surgery system to which the present technique is applicable.

FIG. 15 schematically shows a fourth example of a computer assisted surgery system to which the present technique is applicable.

FIG. 16 schematically shows an example of an arm unit.

FIG. 17 schematically shows an example of a master console.

### DESCRIPTION OF EMBODIMENTS

Referring now to the drawings, wherein like reference numerals designate identical or corresponding parts throughout the several views.

The present disclosure relates to generating a training regime for surgeons that manage stress levels of the surgeon being subject to the training. The surgeon under training may be an inexperienced surgeon or may be an experienced surgeon being trained in an unfamiliar procedure or being trained using different surgical tools or techniques. Typically, this training is carried out in more modern training settings using a surgical simulator.

Although embodiments of the disclosure relates to a training regime for surgeons (and trainee surgeons), the disclosure is not so limited. In other embodiments, stress during surgery may also be applicable to other members of a surgical team, such as an anaesthetist, member of the surgical nursing team or the like. More generally, therefore, embodiments of the disclosure relate to a member of a surgical team.

A surgical simulator is a known system that uses realistic synthetic imagery to train the surgeon. Typically, this imagery is collected from real surgical procedures or from virtually created surgical scenarios.

The disclosure is described in two parts. The first part describes the collection of the surgical data indicating a parameter of a surgical instrument used during a surgical procedure and a stress related parameter of a surgeon performing the surgical procedure and the second part describes the generation of a surgical training simulation based on the surgical data and the stress related parameter collected in the first part.

<First Part>

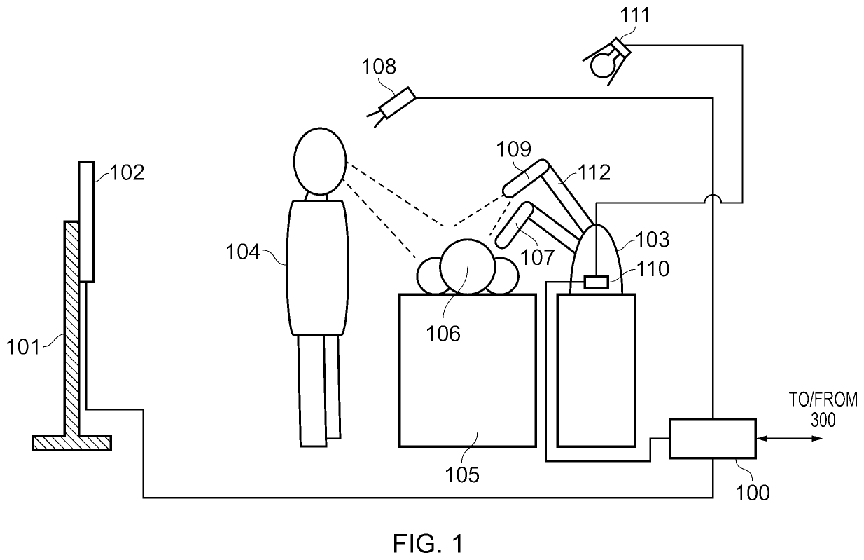

FIG. 1 shows surgery on a patient 106 by an experienced surgeon. The experienced surgeon may be carrying out the surgery (either real life or virtual surgery) without any robotic support (such as “open surgery”), or may be assisted robotic assistance. The robotic assistance may be one or more robotic arm or may include a computer assisted surgical system that may perform an autonomous or semi-autonomous function.

In embodiments of the disclosure, the first and/or second part may include a computer assisted surgical system. In these embodiments, it is especially useful to manage the stress felt by a member of the surgical team as very few members of the surgical team will initially have experience with a computer assisted surgical system and especially in collaborative surgery with a degree of autonomy (such as semiautonomous or fully autonomous where a surgical robot will perform one or more specific tasks autonomously). Moreover, the types of stress felt by the members of the surgical team will likely be different to those felt where no robotic assistance is provided. This makes the training system particularly effective when applied to embodiments where there is an element of computer assistance with the surgery.

During this surgical procedure, the surgical data indicating a parameter of a surgical instrument used during a surgical procedure and a stress related parameter of a surgeon performing the surgical procedure will be collected. The patient 106 lies on an operating table 105 and a human surgeon 104 and a computerised surgical apparatus 103 perform the surgery together. It should be noted here that the surgeon is experienced in the procedure for which the surgical data and the stress related parameter is being collected. In addition, the human surgeon 104 provides an identifier that uniquely identifies him or her.

Each of the human surgeon and computerised surgical apparatus monitor one or more parameters of the surgery, for example, patient data collected from one or more patient data collection apparatuses (e.g. electrocardiogram (ECG) data from an ECG monitor, blood pressure data from a blood pressure monitor, etc.—patient data collection apparatuses are known in the art and not shown or discussed in detail) and one or more parameters determined by analysing images of the surgery (captured by the surgeon's eyes or a camera 109 of the computerised surgical apparatus) or sounds of the surgery (captured by the surgeon's ears or a microphone (not shown) of the computerised surgical apparatus). Each of the human surgeon and computerised surgical apparatus carry out respective tasks during the surgery (e.g. some tasks are carried out exclusively by the surgeon, some tasks are carried out exclusively by the computerised surgical apparatus and some tasks are carried out by both the surgeon and computerised surgical apparatus) and make decisions about how to carry out those tasks using the monitored one or more surgical parameters.

In addition to the parameters of the surgery described above, further surgical data is collected. The surgical data includes movement data of a surgical tool and the surgical robot collected from sensors located within the tool or robot or by tracking the tool or robot and any feedback provided by that tool or robot. For example, sensors include accelerometers, gyroscopes, encoders to measure an angle of a joint or other sensors located within surgical tools such as forceps, tweezers, scalpels, electrodiathermy units or the surgical robot arm that indicates the motion and force of the tool. Moreover, in the example of a surgical robot which is under at least partial control of the experienced surgeon using an interface, the control data provided by the experienced surgeon is also captured.

In addition, image data from cameras showing the experienced surgeon's viewpoint and/or image data from an endoscope or a surgical microscope or an exoscope, or any surgical instrument used in the surgical procedure is captured. This image data may be RGB type image data or may be fluorescent video or the like. In other words, image data of the surgical procedure is image data obtained by the surgical instrument.

In addition, stress related parameters of the experienced surgeon are collected. These stress related parameters may be collected from sensors worn by the surgeon or from images captured of the surgeon or from other physiological parameters of the surgeon. For example, the experienced surgeon may wear a heart rate monitor, sweat analysis sensors, skin conductivity sensors, a breathing rate sensor or a blood pressure monitor. In addition, the experienced surgeon may have his or her blood composition measured regularly or continually during the surgical procedure to measure the amount of cortisol in the blood. The purpose of collecting these stress related parameters is to quantify the stress level of the experienced surgeon at any moment during the surgical procedure. These stress related parameters are captured continually during the surgical procedure.

So, during the surgical procedure, surgical data, stress related parameters and optionally image data are collected. In addition, the unique identifier associated with the experienced surgeon (and possibly the surgical robot) is collected. This information is sent by a communication interface to a network as will be explained later.

Although FIG. 1 shows an open surgery system, the present technique is also applicable to other computer assisted surgery systems where the computerised surgical apparatus (e.g. which holds the medical scope in a computer-assisted medical scope system or which is the slave apparatus in a master-slave system) is able to make decisions which might conflict with the surgeon's decisions. The computerised surgical apparatus is therefore a surgical apparatus comprising a computer which is able to make a decision about the surgery using one or more monitored parameters of the surgery. As a non-limiting example, the computerised surgical apparatus 103 of FIG. 1 is a surgical robot capable of making decisions and undertaking autonomous actions based on images captured by the camera 109.

The robot 103 comprises a controller 110 and one or more surgical tools 107 (e.g. movable scalpel, clamp or robotic hand). The controller 110 is connected to the camera 109 for capturing images of the surgery, to a movable camera arm 112 for adjusting the position of the camera 109 and to adjustable surgical lighting 111 which illuminates the surgical scene and has one or more adjustable lighting parameters such as brightness and colour. For example, the adjustable surgical lighting comprises a plurality of light emitting diodes (LEDs, or laser diodes not shown) of different respective colours. The brightness of each LED is individually adjustable (by suitable control circuitry (not shown) of the adjustable surgical lighting) to allow adjustment of the overall colour and brightness of light output by the LEDs. The controller 110 is also connected to a control apparatus 100. The control apparatus 100 is connected to another camera 108 for capturing images of the surgeon's eyes for use in gaze tracking and to an electronic display 102 (e.g. liquid crystal display or Organic Light Emitting Diode (OLED) display) held on a stand 102 so the electronic display 102 is viewable by the surgeon 104 during the surgery. The control apparatus 100 compares the visual regions of the surgical scene paid attention to by the surgeon 104 and robot 103 to help resolve conflicting surgeon and computer decisions according to the present technique.

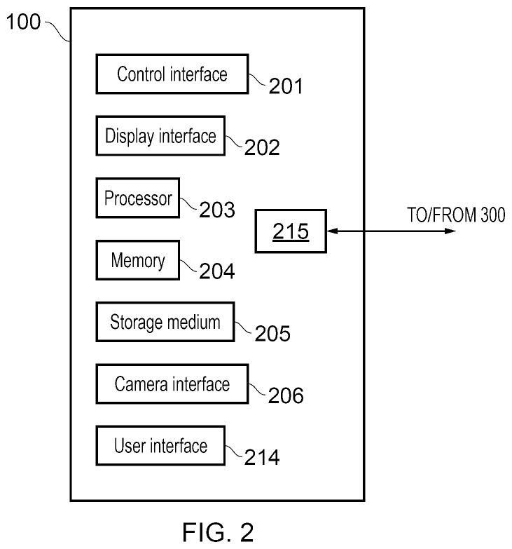

FIG. 2 shows some components of the control apparatus 100.

The control apparatus 100 comprises a control interface 201 for sending electronic information to and/or receiving electronic information from the controller 110, a display interface 202 for sending electronic information representing information to be displayed to the electronic display 102, a processor 203 for processing electronic instructions, a memory 204 for storing the electronic instructions to be processed and input and output data associated with the electronic instructions, a storage medium 205 (e.g. a hard disk drive, solid state drive or the like) for long term storage of electronic information, a camera interface 206 for receiving electronic information representing images of the surgeon's eyes captured by the camera 108 and the image data noted above and a user interface 214 (e.g. comprising a touch screen, physical buttons, a voice control system or the like). Moreover, the communication interface 215 is provided that provides the parameters of the surgery, surgical data, image data, stress related parameters and decision information to the network 300. Each of the control interface 201, display interface 202, processor 203, memory 204, storage medium 205, camera interface 206, user interface 214 and communication interface 215 are implemented using appropriate circuitry, for example. The processor 203 controls the operation of each of the control interface 201, display interface 202, memory 204, storage medium 205, camera interface 206 and user interface 214.

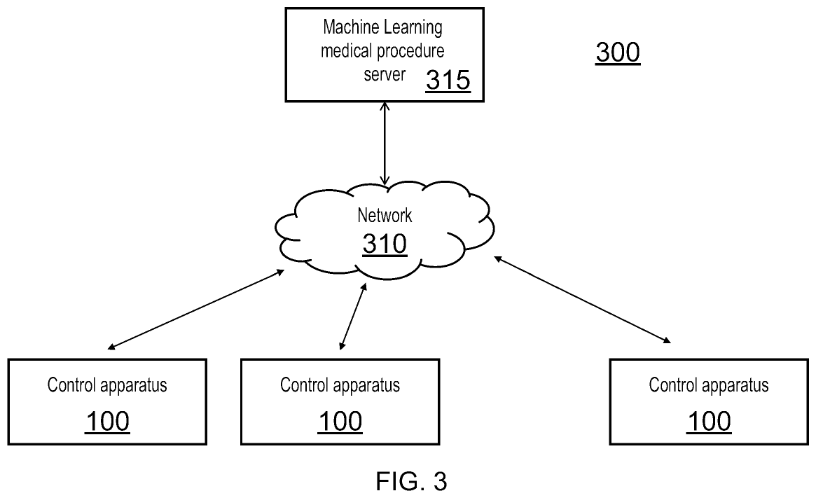

FIG. 3 shows a system 300 according to embodiments of the disclosure. The system 300 includes a plurality of surgical scenarios shown in FIG. 1. In particular, the control apparatus 100 shown in FIG. 1 and FIG. 2 is shown connected to the network 310. The network 310 may be the Internet or another Wide Area Network (WAN) or Local Area Network (LAN). In embodiments, the network 310 may be a Virtual Private Network, such as a network run by a single entity, for example a set of training institutions or a company that hosts surgical training sessions or the like.

Additionally connected to the network 310 is a medical procedure server 315. As will be explained later, the medical procedure server 315 is a computer server that comprises circuitry configured to: receive surgical data indicating a parameter of a surgical instrument used during a surgical procedure and a stress related parameter of a surgeon performing the surgical procedure; and associating the surgical data with the stress related parameter. In addition, the medical procedure server 315 segments the captured image data provided by the control apparatus 100 from the surgery and also generates one or more stress level value that indicate the levels of stress of the experienced surgeon. These stress level values are derived from the stress related parameters captured during the surgery.

It should be noted that the surgical instrument includes one or more of the surgical tool and any one of the cameras in the surgical environment.

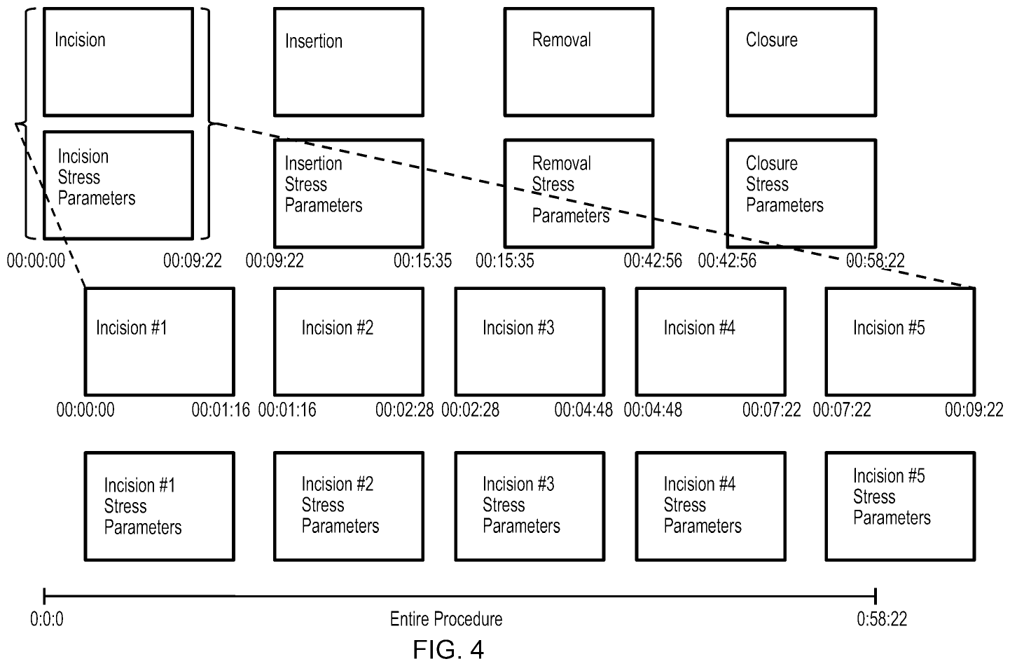

FIG. 4 shows the function of the medical procedure server 315 according to embodiments of the disclosure. In the embodiments of FIG. 4, time units are shown which are denoted using the following nomenclature.

HH:MM:SS

which indicates the number of hours (HH), minutes (MM) and seconds (SS).

The experienced surgeon in the embodiments of FIG. 1 is performing a surgical procedure on a patient. For example, the surgeon may be carrying out a biopsy of a polyp in a patient's colon using an endoscope. The entire procedure has duration of 58 minutes and 22 seconds. This is indicated by the time line toward the bottom of FIG. 4. In the embodiments of FIG. 4, the surgical procedure is split into four sections (sometimes referred to as sub-procedures hereinafter); incision into the patient's colon; insertion of the endoscope; removal of the biopsied polyp and closure of the wound. These four sections may be defined in a pre-surgical plan or may be defined by the surgeon during the surgery. For example, the surgeon may use a voice command when transitioning between the various stages or may provide some other indicator (such as a visual cue) or the like such as being detected based on the state of the surgical tool. This state may include the on/off state of an energy device. Of course, the disclosure is not so limited and the sections may be defined using image recognition such as the images including an anatomical structure or may be defined according to the activities and events within the section are the same (for example, the actions relate to the same procedure such as stemming a bleed, identifying a pathology, closing a wound etc).

In the embodiments of FIG. 4, the medical procedure server 315 splits the image data of the surgical procedure 315 into the four sections. Specifically, the image data captured between 00:00:00 and 00:09:22 are in the incision section; the image data captured between 00:09:22 and 00:15:35 are in the insertion section; the image data captured between 00:15:35 and 00:42:56 are in the removal section; and the image data captured between 00:42:56 and 00:58:22 are in the closure section. Although, in embodiments, the image data captured during the surgery of FIG. 1 is used to generate the virtual surgery simulation, the disclosure is not so limited. In particular, the imagery displayed to the surgeon undergoing training may be synthesised using known techniques. However, it is the use of data indicating a parameter of a surgical instrument used during a surgical procedure and a stress related parameter of a surgeon performing the surgical procedure; and the association of the surgical data with the stress related parameter that allows an appropriate training simulation that avoids trainee surgeon burn-out. It should be noted that the stress related parameter and the surgical data are synchronised in time. In addition, where appropriate, the image data is also synchronised with the stress related parameter and the surgical data.

Of course, the image data may be used to generate a surgical simulation. In the described embodiment, for brevity, the image data is used to generate the surgical training simulation.

As noted above, in addition to the image data, in embodiments, the stress related parameters and the surgical data that are captured during surgery are also split in accordance with the image data. In other words, the stress related parameters and the surgical data associated with each section of the surgery are also defined. For brevity, however, the following disclosure will discuss only stress related parameters.

So, the stress related parameters captured between 00:00:00 and 00:09:22 are in the incision stress parameters; the stress related parameters captured between 00:09:22 and 00:15:35 are in the insertion stress parameters; the stress related parameters captured between 00:15:35 and 00:42:56 are in the removal stress parameters; and the stress related parameters captured between 00:42:56 and 00:58:22 are in the closure stress parameters.

In addition, the medical procedure server 315 may split each of the sections into further sub-sections. This allows for increased granularity of the training regime. In the embodiments shown in FIG. 4, the incision section is further split into five sub-sections (incision #1, incision #2, incision #3, incision #4, incision #5). Accordingly, the stress parameters associated with each sub-section are also defined (incision #1 stress parameters, incision #2 stress parameters, incision #3 stress parameters, incision #4 stress parameters and incision #5 stress parameters).

In the embodiments of FIG. 4, the image data captured between 00:00:00 and 00:01:16 are in the incision #1 subsection; the image data captured between 00:01:16 and 00:02:28 are in the incision #2 subsection; the image data captured between 00:02:28 and 00:04:48 are in the incision #3 subsection, the image data captured between 00:04:48 and 00:07:22 are in the incision #4 subsection and the image data captured between 00:07:22 and 00:09:22 are in the incision #5 subsection. The associated stress parameters are also defined between the respective time segments.

The further sub-sections may contain images relevant to other skills that surgeons require and that may form part of a training regime. For example, although the incision section relates to the experienced surgeon performing an incision into the patient's colon, the incision #2 subsection may include the surgeon performing a cauterisation to stop a bleed. This subsection may be also relevant to a training regime based upon cauterising bleeds.

As noted above, the medical procedure server 315 receives the stress related parameters from the experienced surgeon collected during the surgical procedure. These stress related parameters are used to produce a stress indication value that defines the levels of stress felt by the experienced surgeon during the surgical procedure. In other words, physiological measurements of the experienced surgeon are used to generate an indication of stress felt by the surgeon.

There are a number of different physiological measurements that indicate when a person (such as the experienced surgeon) is under stress as is known. For example, stress levels on various heart rate complexity measures have been investigated in NPL 1 and stress can be estimated based on the overall heart rate and changes to the variability of the heart rate. Moreover, the amount of sweat secreted by a person increases when the person is feeling stress. In addition, the sweat secreted when stressed is provided by the apocrine gland rather than by the eccrine sweat glands which secrete sweat when a person is hot. The apocrine glands are located near dense pockets of hair follicles (such as under the arms, around the groin and on the scalp) and secrete a sweat high in fatty acids and proteins. Therefore, by placing sweat analysis sensors in areas close to the apocrine glands, the sweat (both quantity and composition) associated with stress may be analysed. Finally, and as noted above, the amount of cortisol in the blood of the surgeon indicates whether the surgeon is feeling stressed. Therefore, a rise in cortisol levels within the blood may indicate that the surgeon is feeling under stress. This can be measured by collecting blood from the surgeon continually or at least regularly during the surgical procedure.

One or more of these physiological measurements are used to generate the stress related parameter for the experienced surgeon at a given point in time during the surgical procedure. These physiological measurements may be derived from measurement captured by a wearable device. In embodiments, a single stress related parameter is determined for each section or subsection. Moreover, this single stress related parameter may be determined for each physiological measurement or may be a single stress related parameter for all physiological measurements. The single stress related parameter may be a mean or median average value of the stress or may be the highest value of the stress related parameter over the entire section or sub-section.

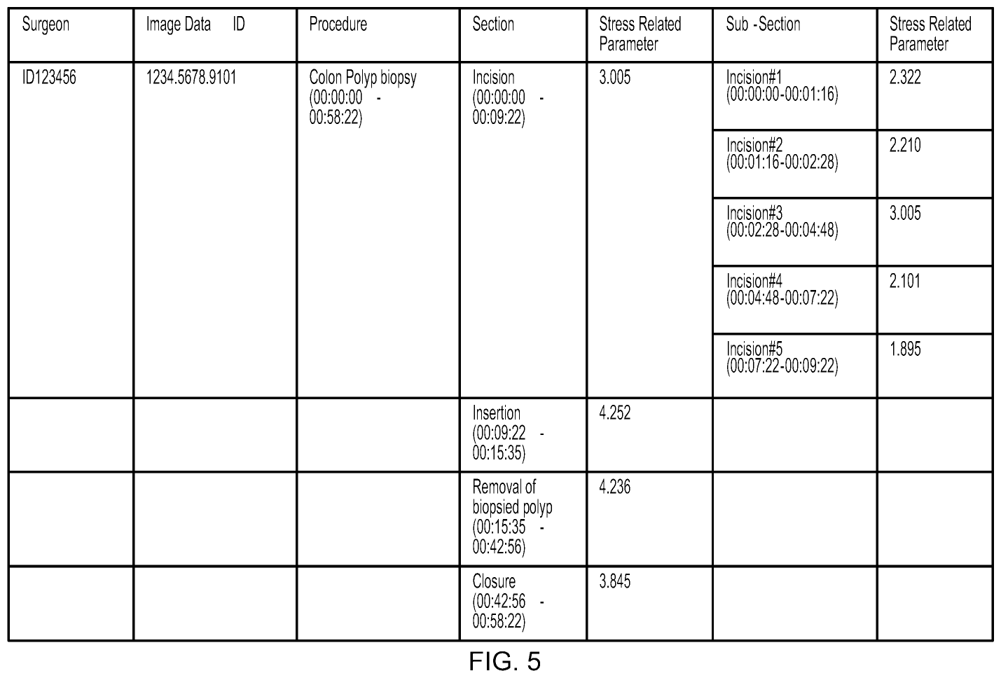

FIG. 5 shows a data structure according to embodiments of the disclosure. The data structure is populated by the medical procedure server 315 after the image data has been split and the stress related parameters for each section and/or sub-section have been determined. As will be apparent from FIG. 5, the unique identifier associated with the experienced surgeon (the “surgeon ID”) is in the data structure. This allows any surgical procedures performed by the experienced surgeon to be searched. This is useful where surgical characteristics of the experienced surgeon are also known. For example, the surgeon may be characterised as a conservative surgeon who performs surgical procedures in a traditional manner or may be characterised as a maverick surgeon who feels more comfortable performing surgical procedures in a less traditional manner. This allows the surgical procedures performed by the surgeon undergoing training to be selected based on the characteristics of the surgeon undergoing training. For example, the surgeon undergoing training may wish to have training by an experienced surgeon who has the same or very different surgical characteristics to himself or herself.

Returning to FIG. 5, an image data identifier (“Image Data ID”) is provided. This is a unique identifier that identifies the captured images from the surgical procedure. In other words, this identifies the complete set of image data from the surgical procedure. As will be explained later, in the embodiments where the image captured during the surgical procedure of FIG. 1 is used to generate the training simulation, when a training simulation for a surgeon under training is created, image data from various surgical procedures will be concatenated together to produce the training simulation. Therefore, uniquely identifying the image data is useful in this regard.

The procedure is identified in the “procedure” section. In this case, a colon polyp biopsy is noted. The duration of the image data associated with this procedure is also noted in the data structure. The naming of the procedure may be selected from a drop down menu or may be entered using free text. In the situation where free text is used to name the procedure, rules associated with a naming convention may be followed to ensure consistency in searching through the procedures.

The sections of the procedure are also noted in the data structure. Moreover, the stress related parameter associated with the section or sub-section is also stored in the data structure. The time period associated with the section or sub-section is noted in the data structure. This is useful in retrieving the relevant image data when the data structure is interrogated looking for a section or sub-section of the image data that provides a certain stress related parameter, surgical data and/or a certain surgical procedure. The relevant section and/or sub-section may be retrieved easily.

Additionally, although not shown in FIG. 5, the surgical data collected during the surgical procedure is stored in the data structure. This will be compared with the surgical data collected during the same section or sub-section of the training regime to determine whether the surgeon undergoing training is performing the section or subsection correctly. In other words, the same surgical data is collected from the surgeon undergoing training as was collected from the experienced surgeon for the various sections of the surgical procedure.

To summarise the first part, according to embodiments, there is provided a device, comprising circuitry configured to: receive surgical data indicating a parameter of a surgical instrument used during a surgical procedure and a stress related parameter of a surgeon performing the surgical procedure; and associating the surgical data with the stress related parameter. This allows a training simulation to be created that tests the surgical skill of the surgeon undergoing training by providing the surgical data whilst ensuring the surgeon does not have burn-out by managing the stress levels of the surgeon undergoing training.

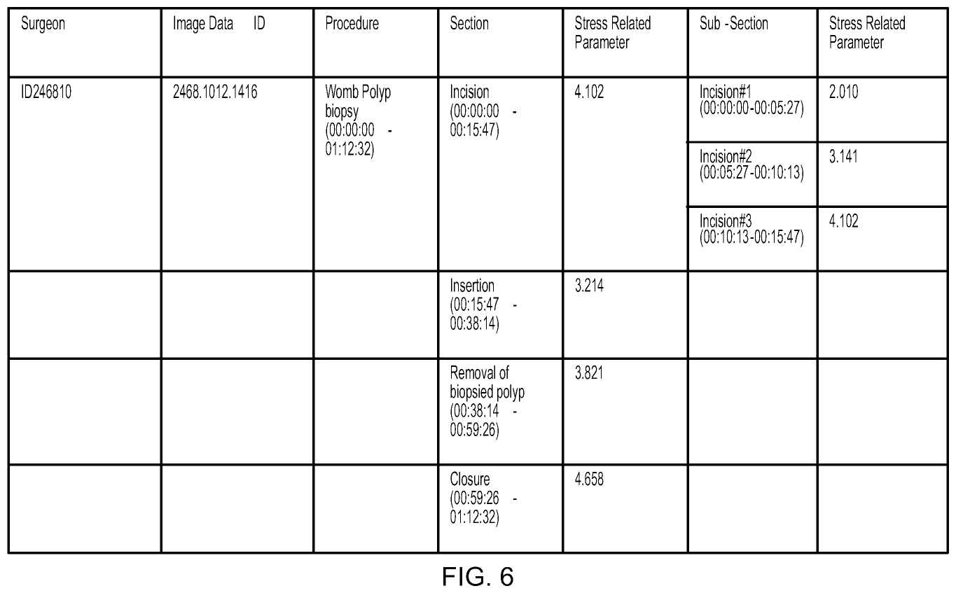

FIG. 6 shows a data structure of a different surgical procedure carried out by a different expert surgeon according to embodiments of the disclosure. As will be appreciated, the population of the data structure of FIG. 6 is using the same technique as explained with reference to FIG. 5. In other words, a different expert surgeon carries out a different surgical procedure and the same information that was captured and stored in respect of the data structure of FIG. 5 is also captured in respect of FIG. 6. In the example of FIG. 6, a polyp is removed from a patient's womb. Of course, other surgical procedures are envisaged and moreover, the same or different procedures may be captured. These same or different procedures may be carried out by the same or different surgeons.

As will be appreciated, the procedure of FIG. 6 is split into the same four sections as those for the procedure of FIG. 5; specifically, incision, insertion of the endoscope, removal of the biopsied polyp and closure. However, the stress associated with each of the sections is different to the stress associated with the same section in FIG. 5.

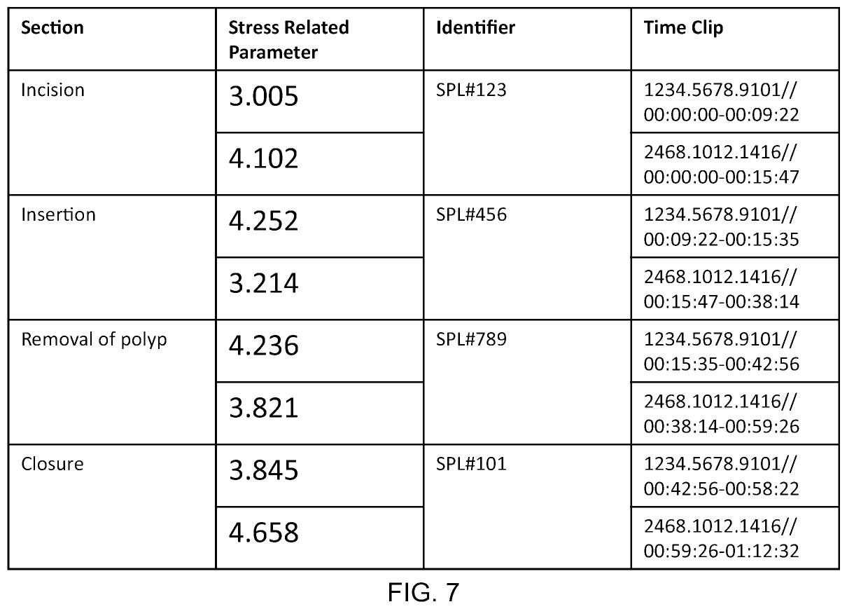

FIG. 7 shows a second data structure according to embodiments of the disclosure. The second data structure groups the sections (and/or sub-sections if desired) into categories. In the embodiments of FIG. 7, the sections of the data structure of FIG. 5 and FIG. 6 are grouped together. Specifically, as both data structures include sections having incision, insertion of an endoscope, removal of a polyp and closure parts, the second data structure groups the sections into these categories. Of course, the disclosure is not so limited and the second data structure may group the sections into any category. For example, the second data structure may be grouped according to surgeon ID or characteristic of surgeon, stress related parameter or the like.

Moreover, the mechanism for grouping the categories is not limited in the disclosure. The grouping of the sections may be achieved by reviewing the name of the section in the data structures of FIG. 5 and FIG. 6 so that sections having the same or similar names are grouped together. This may be done automatically using text recognition or manually.

In addition to the sections from the two data structures of FIG. 5 and FIG. 6 being grouped together, each group in the embodiments of FIG. 7 are provided with a unique identifier. This unique identifier is thus associated with the group and each section within the group. In the embodiments of FIG. 7, the incision group is provided the identifier “SPL #123”, the insertion group is provided the identifier “SPL #456”, the removal of polyp group is provided the identifier “SPL #789” and the closure group is provided the identifier “SPL #101”. The functionality of the identifier may be provided by the name of section. In other words, the column “identifier” is optional.

The “stress related parameter” value for each section is stored in the second data structure of FIG. 7. In other words, the stress related parameter value for each section in the data structure of FIG. 5 and FIG. 6 is stored in second data structure of FIG. 7 in the relevant group. So, the stress related parameter of the incision section of FIG. 5 and the stress related parameter of the incision section of FIG. 6 are stored in the incision group. Moreover, the “time clip” is also stored in association with the relevant stress related parameter. The time clip is a unique identifier that uniquely identifies the video clip of the particular section. The time clip identifier may be a Unique Material Identifier (UMID) or may be any kind of unique identifier that allows the video clip of the surgery to be retrieved. In the example of FIG. 7, the image data ID is used in conjunction with the time units during which the relevant section was captured. So, in the example of the incision section from FIG. 5, the time clip identifier is:

1234.5678.9101//00:00:00-00:09:22
Which is formed from the image data ID (1234.5678.9101) and the time unit during which the incision section was captured (00:00:00-00:09:22).

The purpose of the second data structure of FIG. 7 is to allow the video footage of a particular section of a procedure having a particular stress related parameter to be retrieved and to be shown to a surgeon undergoing training in creating the surgical training simulation. Of course, retrieval of the image is not necessary in other embodiments. In other words, the purpose of the second data structure of FIG. 7 is to allow the surgeon undergoing training to practice various procedures; each of the same procedure having a different stress level. The second data structure of FIG. 7 will be used to assist in the training of surgeon as will now be explained as it will manage the stress levels felt by the surgeon undergoing training.

In embodiments, the surgical procedure is formed of a plurality of subprocedures, and the circuitry is configured to divide the surgical procedure into a plurality of sections, each section comprising the surgical data and the stress related parameter relating to a different subprocedure.

This allows the training simulator to select relevant parts of different surgical procedures together so that the surgeon undergoing training may conduct a relevant surgical procedure that has the correct stress levels.

The circuitry may be configured to receive image data of the surgical procedure and the image data of the surgical procedure is image data obtained by the surgical instrument.

The surgical data may include movement data of a robotic surgical instrument. Further, the circuitry may be configured to receive the stress related parameter from a wearable device that is worn by a member in the surgical team, such as one or more of the surgeons.

To summarise the first part, according to embodiments, there is provided a device, comprising circuitry configured to: receive surgical data indicating a parameter of a surgical instrument used during a surgical procedure and a stress related parameter of a member of a surgical team performing the surgical procedure; and associating the surgical data with the stress related parameter. This allows a training simulation to be created that tests the surgical skill of the surgeon undergoing training by providing the surgical data whilst ensuring the surgeon does not have burn-out by managing the stress levels of the surgeon undergoing training.

<Second Part>

A training regime for a surgeon undergoing training will now be described.

Firstly, it is envisaged that the surgeon undergoing training will collect the same surgical data and stress related parameters as the experienced surgeon carrying out the surgery in FIG. 1. It is envisaged that the training will be carried out in a known surgical training environment. Such a training environment will therefore not be described for brevity. The surgical training environment may use the images captured of the procedure carried out by the experienced surgeon or may use a virtual reality environment. The surgical training regime will be generated using the stress related parameters and the surgical data captured during the surgery explained in reference to FIG. 1. It should be noted that whilst the arrangement of such a training environment is known, the data used to formulate the training regime is not.

As noted above, the second part of the disclosure provides a surgical training apparatus comprising circuitry configured to: receive the surgical data and the associated stress related parameter from the device of the first part; and generate a surgical training simulation based on the surgical data and the stress related parameter.

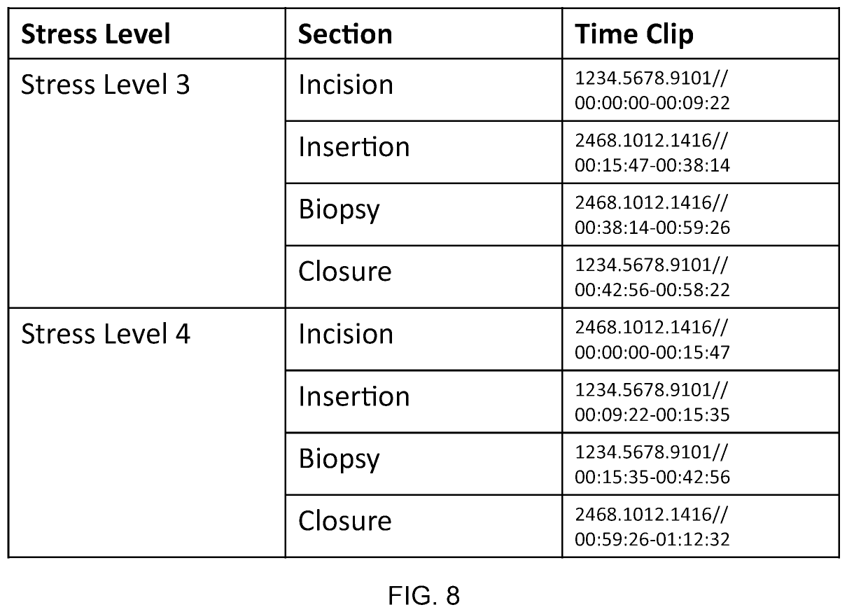

FIG. 8 shows a training schedule for a simulation that manages the stress levels of the surgeon undergoing training according to embodiments. The training schedule is designed to train the surgeon to carry out a procedure such as removal of a polyp using an endoscope. However, the training schedule manages the stress level experienced by the surgeon undergoing training at each different part of the procedure.

As noted above in respect of the surgery carried out by the experienced surgeon, the procedure to remove a polyp using an endoscope in the training regime is broadly comprised of four sections (or subprocedures); the incision section where an incision is performed on the patient to allow entry of the endoscope; an insertion section where the endoscope is inserted into the patient; a biopsy section where a biopsy of the polyp is carried out and finally a closure section where the endoscope is removed, the patient closed and the surgery finished.

In the training schedule of FIG. 8, the surgeon undergoing training will perform the same sections. That is, during training, the surgeon undergoing training will perform the incision section, the insertion section, the biopsy section and the closure section. However, the sections that the surgeon undergoing training will perform during any one training procedure will be determined by the stress level associated with the section.

In the example, of FIG. 8, therefore, the surgeon undergoing training will firstly perform the sections having the lowest stress level (Stress Level 3) and at an appropriate time, the surgeon undergoing training will then perform the sections having higher stress levels. The appropriate time in embodiments, is when the surgeon undergoing training can perform the procedure at a stress level that is similar to the experienced surgeon. In other words, the stress related parameter of the surgeon undergoing training is compared with the stress related parameter of the experienced surgeon and when that comparison indicates similar levels of stress induced in both the experienced surgeon and the surgeon undergoing training is similar then the surgeon undergoing training is allowed to progress to the section or subprocedure that induces a higher level of stress in the experienced surgeon. Of course, the quality of the surgery performed by the surgeon undergoing training would need to be similar to that performed by the experienced surgeon as well in order for the surgeon undergoing training to be deemed to have mastered a particular section. In order to determine the quality of the surgery, the surgical data captured during the surgical procedure carried out by the experienced surgeon is compared with the surgical data captured during the surgical procedure carried out by the surgeon undergoing training. In the event that the surgical data is comparable (i.e. similar), then the surgeon under training is deemed to have performed the surgical procedure to an appropriate quality level. In other words, if the surgical data captured from the surgeon undergoing training is different to that of the experienced surgeon by less than a threshold amount, or the data differs only in immaterial aspects, then the surgeon undergoing training is deemed to have achieved the required quality. Accordingly, if the surgeon undergoing training achieves the requisite quality whilst having similar levels of stress as the experienced surgeon, the section is deemed mastered and the surgeon undergoing training may move on to a more stressful and/or complicated section.

In the specific embodiment of FIG. 8, the sections in the “Stress Level 3” column are sections that have a stress level of 3 and the sections in the “Stress Level 4” column are sections that have a stress level of 4. In particular, in the column “Stress Level 3”, the incision and closure sections are taken from the data structure of FIG. 5 and the insertion and biopsy sections are taken from the data structure of FIG. 6. These sections have a stress level induced in the experienced surgeon of around 3. Conversely, in the “column “Stress Level 4”, the incision and closure sections are taken from the data structure of FIG. 6 and the insertion and biopsy sections are taken from the data structure of FIG. 5. These sections have a stress level induced in the experienced surgeon of around 4. Accordingly, the surgeon undergoing training will begin on the lower stress rated program (Stress Level 3) and once this is passed by the surgeon undergoing training, the surgeon undergoing training will then move to the higher stress rated program (Stress Level 4). This means that the surgeon undergoing training will be taught how to perform various surgical procedures whilst managing the stress levels of the surgeon undergoing training.

Although the foregoing in FIG. 8 describes providing a simulation of an entire procedure, the disclosure is not so limited. For example, a simulation of only part of the surgery can be provided with the training simulation started at some arbitrary point just prior to the section. In instances, it may be necessary for part of the previous or subsequent section in the procedure to be carried out by the surgeon undergoing training for continuity or coherence within the simulation. In this case, other sections, sub-sections and/or parts of sections or sub-sections would need to be simulated.

Although the foregoing describes one mechanism for generating a simulation, the disclosure is not so limited.

Firstly, in respect of the data structures of FIGS. 5 and 6, further metrics may be established. In the embodiments of FIG. 5 and FIG. 6, the “Stress Related Parameter” is found for each section or sub-section. As noted above, this may be the highest or average value of stress exhibited by the experienced surgeon during a section or subsection. In addition to the “Stress Related Parameter”, a “Stress Relationship Value” (SRV) may also be found. The SRV is a measure of the relationship between a particular section or sub-section and the stress levels it induces in surgeons. In particular, the SRV has two functions: 1) to find an average of the stress levels experienced for each section or sub-section with the same unique identifier (such as SPL #123 and the like) across all the experienced surgeons; and 2) to find the correlation of stress levels between all the pairs of different sections (in other words, those sections or sub-sections that have different SPL values).

So, in the example embodiments of FIG. 5 and FIG. 6, to find an average of the stress levels experienced for each section or sub-section, two surgeons performed a polyp biopsy and the mean average value for each section within the procedure is shown below in Table 1.

TABLE 1

| Section | Mean Stress Related Parameter |
|---|---|
| Incision | 3.554 |
| Insertion | 3.733 |
| Removal of Polyp | 4.029 |
| Closure | 4.252 |

Although a mean value is established, the disclosure is not so limited and any kind of average value (such as the median average) is envisaged. By calculating the average value for the SRV across a plurality (which may be all or a subset) of the experienced surgeons who have carried out the procedure, a better representation of the stress levels associated with the section or sub-section for an experienced surgeon may be obtained.

The average value may then be used to calculate a stress correlation value between different sections. This allows a value to be determined that indicates how much one section induces similar levels of increased or decreased stress in different experienced surgeons, relative to the average SRV across experienced surgeons for that section or sub-section.

The stress correlation value may, in embodiments, be calculated in the following manner:
a. Each section identifier (SPL number) is given a vector with a value in the vector for each experienced surgeon identifier filled with the Mean Stress Related Parameter for that section identifier.
i. The vectors are normalized by, for example, dividing the vector by its mean value.

b. Across all the section identifier a correlation analysis is performed using these vectors to determine the similarity of these vectors.
i. In this way two sections or sub-sections which both are stressful in one set of surgeons but not in another set of surgeons are found.

c. This process yields, for each pair of section identifiers, a Section Correlation Link Value ranging from 0 to 1.

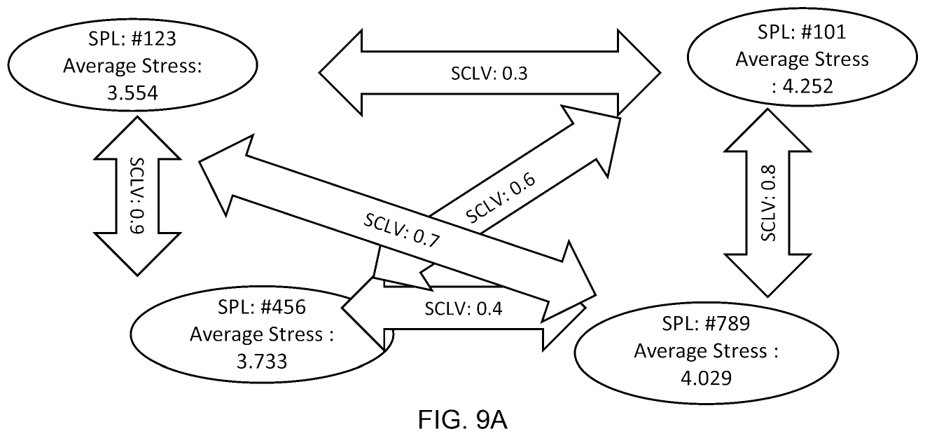

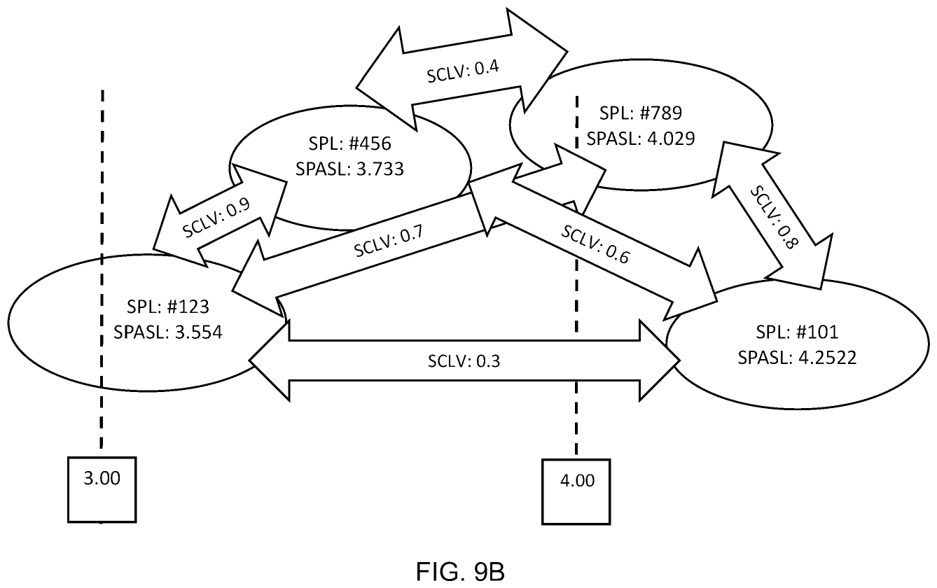

Referring to FIGS. 9A and 9B a training graph according to embodiments is shown. The training graph may be used to assist in provision of training for the surgeon.

Referring to FIG. 9A, the section identifier (SPL number) is a node in each graph. The nodes are ordered in one dimension (in this case from left to right) according to their Average Stress level. The nodes are connected by edges given the value by the Section Correlation Link Value (SCLV).

Referring to FIG. 9B, the training graph is created using the following process.

a. A first virtual training program is constructed using the training graph, by choosing an initial level of section average stress value as a threshold value. All the nodes to the left of that line are marked as ‘in training’. Virtual surgery simulations are then generated by the surgical simulation server which will play through those section identifier which are ‘in training’ while avoiding those section identifier which are not ‘in training’. Each simulation generates the average stress level for each point in the simulation.

b. When a surgeon undergoing training performs these virtual surgical training sessions, the same sensors are used to collect stress related parameters but in this case for the surgeon undergoing training. The levels of stress in this trainee simulation data is compared to the stress related parameters derived from the experienced surgeon. When the stress levels of the surgeon undergoing training within the section with a specific section label are close enough to the stress related parameters of the experienced surgeon, and that the surgical data is similar to those of the experienced surgeon so the quality of the surgery is satisfactory, then that node in the graph is labelled as ‘mastered’.

c. For each node that is labelled as ‘mastered’, nodes to the right in the Training Graph (those with higher average stress level) are examined to determine if they should be ‘in training’. The values of the average stress level of mastered sections and sections linked to it and the value of the Section Correlation Link Value (SCLV) between them (and possibly the current the average stress levels for that section) and uses them to calculate if it should return ‘True’ or ‘False’. If ‘True’ the node linked to the ‘mastered’ node is set as ‘in training’. For example a node which has a high SCLV and whose average stress level for that section is not too much higher would return ‘True’.

d. A subsequent training program of Virtual surgery simulations are then generated by the surgical simulator which will play through those section identifier nodes which are ‘in training’ while avoiding those section identifier nodes which are not ‘in training’. The probability of the use of section identifier nodes which are marked as ‘mastered’ is reduced so that the training concentrates on new sections which are ‘in training’ but not ‘mastered’.

e. This process of ‘mastering’ certain sections and extending the sections which are ‘in training’ then continues until the trainee has ‘mastered’ all the sections considered to be important in their training.

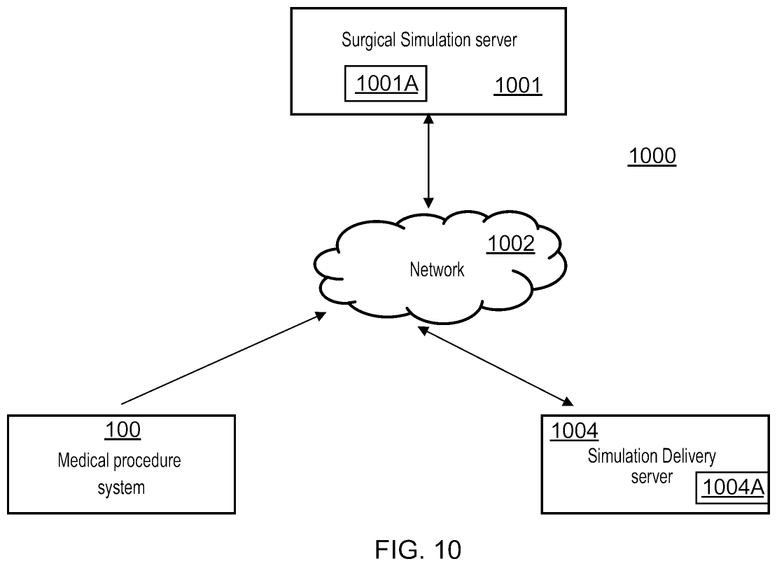

FIG. 10 shows a system 1000 according to embodiments of the disclosure. The system 1000 includes a network 1002 which may be a Wide Area Network (WAN), Local Area Network (LAN), Internet or the like. A surgical simulation server 1001 which generates the training simulation of FIGS. 9A and 9B is connected to the network 1002. The surgical simulation server 1001 includes circuitry 1001A that is configured to perform embodiments of the 2^{nd} part described above. The circuitry 1001A may include processing circuitry such as a microprocessor that uses software stored in a storage medium (not shown) to operate, Communication circuitry may also be provided within the circuitry 1001A to communicate with the network 1002.

Further, the control unit 100 and a simulation delivery server 1004 is connected to the network 1002. The simulation delivery server 1004 may include circuitry 1004A that is configured to perform embodiments of the 1st part described above. The circuitry 1004A may include processing circuitry such as a microprocessor that uses software stored in a storage medium (not shown) to operate, Communication circuitry may also be provided within the circuitry 1004A to communicate with the network 1002.

In summary, the 2^{nd} part discloses a surgical training apparatus comprising circuitry configured to: receive the surgical data and the associated stress related parameter from the device of any one of embodiments of the 1^{st} part; and generate a surgical training simulation based on the surgical data and the stress related parameter.

As noted above, by using the surgical data and the associated stress related parameter, a surgeon undergoing training can master the skills of becoming a surgeon without risking burnout by managing the stress levels of the surgeon undergoing training.

The circuitry may be further configured to: receive a second stress related parameter from the surgeon using the surgical training apparatus; and generate the surgical training simulation based on the second stress related parameter.

The circuitry may be further configured to: generate the surgical training simulation based on a value of the stress related parameter that is higher than the second stress related parameter. This allows the surgeon undergoing training to progress through his or her training to more complex matters where stress levels increase in a more gradual manner. This reduces the risk of burnout.

The circuitry may be configured to: control a display to display the generated surgical training simulation.

The circuitry may be configured to control the display to display a training graph defining the surgical training simulation.

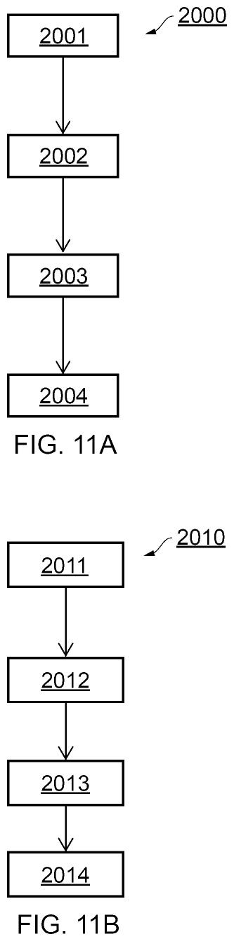

FIG. 11A shows embodiments of the 1^{st} part of the disclosure. Specifically, FIG. 11A shows embodiments of the 1^{st} part which are carried out by the circuitry 1004A in the simulation delivery server 1004 (the device). The process 2000 begins at step 2001. The process moves to step 2002 where surgical data indicating a parameter of a surgical instrument used during a surgical procedure and a stress related parameter of a surgeon performing the surgical procedure are received. The process moves to step 2003 where the surgical data is associated with the stress related parameter. The process moves to step 2004 where the process ends.

FIG. 11B shows embodiments of the 2^{nd} part of the disclosure. Specifically, FIG. 11B shows embodiments of the 2^{nd} part which are carried out by the circuitry 1001A in the surgical simulation server 1001 (the surgical training apparatus). The process 2010 begins at step 2011. The process moves to step 2012 where receive the surgical data and the associated stress related parameter from the device of the 1^{st} part is received. The process then moves to step 2013 where a surgical training simulation based on the surgical data and the stress related parameter is generated. The process then moves to step 2014 where the process ends.

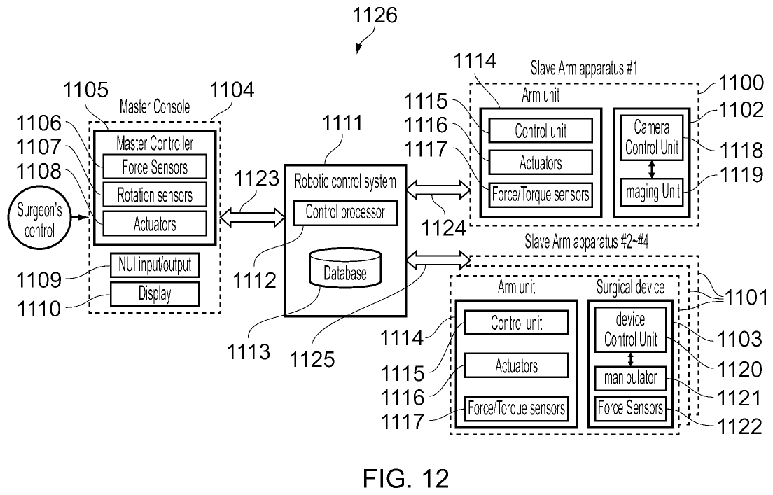

FIG. 12 schematically shows an example of a computer assisted surgery system 1126 to which the present technique may be applicable. The computer assisted surgery system is a master-slave system incorporating an autonomous arm 1100 and one or more surgeon-controlled arms 1101. The autonomous arm holds an imaging device 1102 (e.g. a surgical camera or medical vision scope such as a medical endoscope, surgical microscope or surgical exoscope). The one or more surgeon-controlled arms 1101 each hold a surgical device 1103 (e.g. a cutting tool or the like). The imaging device of the autonomous arm outputs an image of the surgical scene to an electronic display 1110 viewable by the surgeon. The autonomous arm autonomously adjusts the view of the imaging device whilst the surgeon performs the surgery using the one or more surgeon-controlled arms to provide the surgeon with an appropriate view of the surgical scene in real time.

The surgeon controls the one or more surgeon-controlled arms 1101 using a master console 1104. The master console includes a master controller 1105. The master controller 1105 includes one or more force sensors 1106 (e.g. torque sensors), one or more rotation sensors 1107 (e.g. encoders) and one or more actuators 1108. The master console includes an arm (not shown) including one or more joints and an operation portion. The operation portion can be grasped by the surgeon and moved to cause movement of the arm about the one or more joints. The one or more force sensors 1106 detect a force provided by the surgeon on the operation portion of the arm about the one or more joints. The one or more rotation sensors detect a rotation angle of the one or more joints of the arm. The actuator 1108 drives the arm about the one or more joints to allow the arm to provide haptic feedback to the surgeon. The master console includes a natural user interface (NUI) input/output for receiving input information from and providing output information to the surgeon. The NUI input/output includes the arm (which the surgeon moves to provide input information and which provides haptic feedback to the surgeon as output information). The NUI input/output may also include voice input, line of sight input and/or gesture input, for example. The master console includes the electronic display 1110 for outputting images captured by the imaging device 1102.

The master console 1104 communicates with each of the autonomous arm 1100 and one or more surgeon-controlled arms 1101 via a robotic control system 1111. The robotic control system is connected to the master console 1104, autonomous arm 1100 and one or more surgeon-controlled arms 1101 by wired or wireless connections 1123, 1124 and 1125. The connections 1123, 1124 and 1125 allow the exchange of wired or wireless signals between the master console, autonomous arm and one or more surgeon-controlled arms.

The robotic control system includes a control processor 1112 and a database 1113. The control processor 1112 processes signals received from the one or more force sensors 1106 and one or more rotation sensors 1107 and outputs control signals in response to which one or more actuators 1116 drive the one or more surgeon controlled arms 1101. In this way, movement of the operation portion of the master console 1104 causes corresponding movement of the one or more surgeon controlled arms.

The control processor 1112 also outputs control signals in response to which one or more actuators 1116 drive the autonomous arm 1100. The control signals output to the autonomous arm are determined by the control processor 1112 in response to signals received from one or more of the master console 1104, one or more surgeon-controlled arms 1101, autonomous arm 1100 and any other signal sources (not shown). The received signals are signals which indicate an appropriate position of the autonomous arm for images with an appropriate view to be captured by the imaging device 1102. The database 1113 stores values of the received signals and corresponding positions of the autonomous arm.

For example, for a given combination of values of signals received from the one or more force sensors 1106 and rotation sensors 1107 of the master controller (which, in turn, indicate the corresponding movement of the one or more surgeon-controlled arms 1101), a corresponding position of the autonomous arm 1100 is set so that images captured by the imaging device 1102 are not occluded by the one or more surgeon-controlled arms 1101.

As another example, if signals output by one or more force sensors 1117 (e.g. torque sensors) of the autonomous arm indicate the autonomous arm is experiencing resistance (e.g. due to an obstacle in the autonomous arm's path), a corresponding position of the autonomous arm is set so that images are captured by the imaging device 1102 from an alternative view (e.g. one which allows the autonomous arm to move along an alternative path not involving the obstacle).

It will be appreciated there may be other types of received signals which indicate an appropriate position of the autonomous arm.

The control processor 1112 looks up the values of the received signals in the database 1112 and retrieves information indicating the corresponding position of the autonomous arm 1100. This information is then processed to generate further signals in response to which the actuators 1116 of the autonomous arm cause the autonomous arm to move to the indicated position.

Each of the autonomous arm 1100 and one or more surgeon-controlled arms 1101 includes an arm unit 1114. The arm unit includes an arm (not shown), a control unit 1115, one or more actuators 1116 and one or more force sensors 1117 (e.g. torque sensors). The arm includes one or more links and joints to allow movement of the arm. The control unit 1115 sends signals to and receives signals from the robotic control system 1111.

In response to signals received from the robotic control system, the control unit 1115 controls the one or more actuators 1116 to drive the arm about the one or more joints to move it to an appropriate position. For the one or more surgeon-controlled arms 1101, the received signals are generated by the robotic control system based on signals received from the master console 1104 (e.g. by the surgeon controlling the arm of the master console). For the autonomous arm 1100, the received signals are generated by the robotic control system looking up suitable autonomous arm position information in the database 1113.

In response to signals output by the one or more force sensors 1117 about the one or more joints, the control unit 1115 outputs signals to the robotic control system. For example, this allows the robotic control system to send signals indicative of resistance experienced by the one or more surgeon-controlled arms 1101 to the master console 1104 to provide corresponding haptic feedback to the surgeon (e.g. so that a resistance experienced by the one or more surgeon-controlled arms results in the actuators 1108 of the master console causing a corresponding resistance in the arm of the master console). As another example, this allows the robotic control system to look up suitable autonomous arm position information in the database 1113 (e.g. to find an alternative position of the autonomous arm if the one or more force sensors 1117 indicate an obstacle is in the path of the autonomous arm).

The imaging device 1102 of the autonomous arm 1100 includes a camera control unit 1118 and an imaging unit 1119. The camera control unit controls the imaging unit to capture images and controls various parameters of the captured image such as zoom level, exposure value, white balance and the like. The imaging unit captures images of the surgical scene. The imaging unit includes all components necessary for capturing images including one or more lenses and an image sensor (not shown). The view of the surgical scene from which images are captured depends on the position of the autonomous arm.

The surgical device 1103 of the one or more surgeon-controlled arms includes a device control unit 1120, manipulator 1121 (e.g. including one or more motors and/or actuators) and one or more force sensors 1122 (e.g. torque sensors).

The device control unit 1120 controls the manipulator to perform a physical action (e.g. a cutting action when the surgical device 1103 is a cutting tool) in response to signals received from the robotic control system 1111. The signals are generated by the robotic control system in response to signals received from the master console 1104 which are generated by the surgeon inputting information to the NUI input/output 1109 to control the surgical device. For example, the NUI input/output includes one or more buttons or levers comprised as part of the operation portion of the arm of the master console which are operable by the surgeon to cause the surgical device to perform a predetermined action (e.g. turning an electric blade on or off when the surgical device is a cutting tool).

The device control unit 1120 also receives signals from the one or more force sensors 1122. In response to the received signals, the device control unit provides corresponding signals to the robotic control system 1111 which, in turn, provides corresponding signals to the master console 1104. The master console provides haptic feedback to the surgeon via the NUI input/output 1109. The surgeon therefore receives haptic feedback from the surgical device 1103 as well as from the one or more surgeon-controlled arms 1101. For example, when the surgical device is a cutting tool, the haptic feedback involves the button or lever which operates the cutting tool to give greater resistance to operation when the signals from the one or more force sensors 1122 indicate a greater force on the cutting tool (as occurs when cutting through a harder material, e.g. bone) and to give lesser resistance to operation when the signals from the one or more force sensors 1122 indicate a lesser force on the cutting tool (as occurs when cutting through a softer material, e.g. muscle). The NUI input/output 1109 includes one or more suitable motors, actuators or the like to provide the haptic feedback in response to signals received from the robot control system 1111.

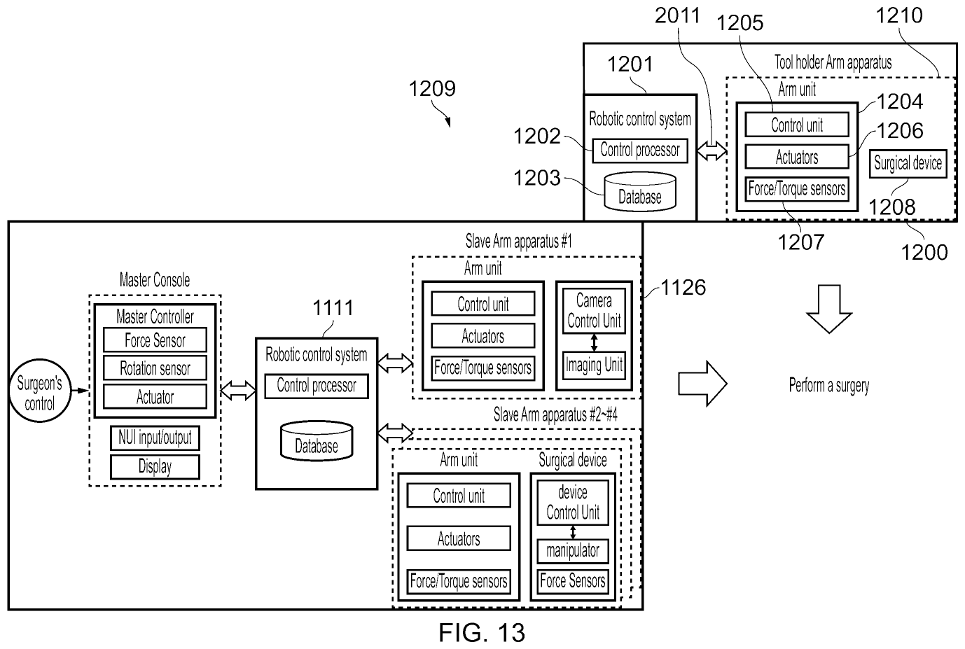

FIG. 13 schematically shows another example of a computer assisted surgery system 1209 to which the present technique is applicable. The computer assisted surgery system 1209 is a surgery system in which the surgeon performs tasks via the master-slave system 1126 and a computerised surgical apparatus 1200 performs tasks autonomously.

The master-slave system 1126 is the same as FIG. 5 and is therefore not described. The master-slave system may, however, be a different system to that of FIG. 5 in alternative embodiments or may be omitted altogether (in which case the system 1209 works autonomously whilst the surgeon performs conventional surgery).

The computerised surgical apparatus 1200 includes a robotic control system 1201 and a tool holder arm apparatus 1210. The tool holder arm apparatus 1210 includes an arm unit 1204 and a surgical device 1208. The arm unit includes an arm (not shown), a control unit 1205, one or more actuators 1206 and one or more force sensors 1207 (e.g. torque sensors). The arm includes one or more joints to allow movement of the arm. The tool holder arm apparatus 1210 sends signals to and receives signals from the robotic control system 1201 via a wired or wireless connection 1211. The robotic control system 1201 includes a control processor 1202 and a database 1203. Although shown as a separate robotic control system, the robotic control system 1201 and the robotic control system 1111 may be one and the same. The surgical device 1208 has the same components as the surgical device 1103. These are not shown in FIG. 6.

In response to control signals received from the robotic control system 1201, the control unit 1205 controls the one or more actuators 1206 to drive the arm about the one or more joints to move it to an appropriate position. The operation of the surgical device 1208 is also controlled by control signals received from the robotic control system 1201. The control signals are generated by the control processor 1202 in response to signals received from one or more of the arm unit 1204, surgical device 1208 and any other signal sources (not shown). The other signal sources may include an imaging device (e.g. imaging device 1102 of the master-slave system 1126) which captures images of the surgical scene. The values of the signals received by the control processor 1202 are compared to signal values stored in the database 1203 along with corresponding arm position and/or surgical device operation state information. The control processor 1202 retrieves from the database 1203 arm position and/or surgical device operation state information associated with the values of the received signals. The control processor 1202 then generates the control signals to be transmitted to the control unit 1205 and surgical device 1208 using the retrieved arm position and/or surgical device operation state information.

For example, if signals received from an imaging device which captures images of the surgical scene indicate a predetermined surgical scenario (e.g. via neural network image classification process or the like), the predetermined surgical scenario is looked up in the database 1203 and arm position information and/or surgical device operation state information associated with the predetermined surgical scenario is retrieved from the database. As another example, if signals indicate a value of resistance measured by the one or more force sensors 1207 about the one or more joints of the arm unit 1204, the value of resistance is looked up in the database 1203 and arm position information and/or surgical device operation state information associated with the value of resistance is retrieved from the database (e.g. to allow the position of the arm to be changed to an alternative position if an increased resistance corresponds to an obstacle in the arm's path). In either case, the control processor 1202 then sends signals to the control unit 1205 to control the one or more actuators 1206 to change the position of the arm to that indicated by the retrieved arm position information and/or signals to the surgical device 1208 to control the surgical device 1208 to enter an operation state indicated by the retrieved operation state information (e.g. turning an electric blade to an “on” state or “off” state if the surgical device 1208 is a cutting tool).

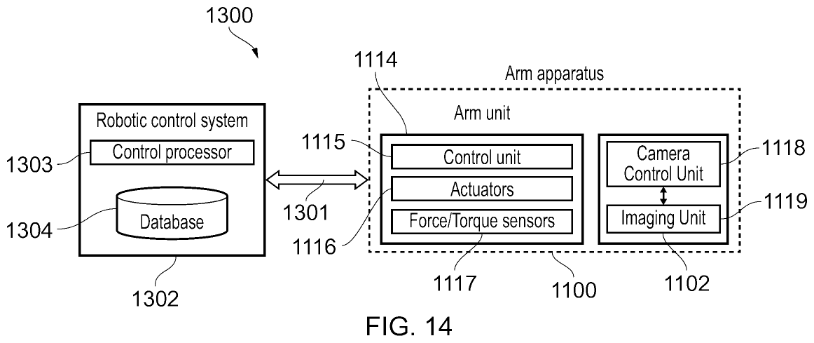

FIG. 14 schematically shows another example of a computer assisted surgery system 1300 to which the present technique is applicable. The computer assisted surgery system 1300 is a computer assisted medical scope system in which an autonomous arm 1100 holds an imaging device 1102 (e.g. a medical scope such as an endoscope, microscope or exoscope). The imaging device of the autonomous arm outputs an image of the surgical scene to an electronic display (not shown) viewable by the surgeon. The autonomous arm autonomously adjusts the view of the imaging device whilst the surgeon performs the surgery to provide the surgeon with an appropriate view of the surgical scene in real time. The autonomous arm 1100 is the same as that of FIG. 12 and is therefore not described. However, in this case, the autonomous arm is provided as part of the standalone computer assisted medical scope system 1300 rather than as part of the master-slave system 1126 of FIG. 12. The autonomous arm 1100 can therefore be used in many different surgical setups including, for example, laparoscopic surgery (in which the medical scope is an endoscope) and open surgery.

The computer assisted medical scope system 1300 also includes a robotic control system 1302 for controlling the autonomous arm 1100. The robotic control system 1302 includes a control processor 1303 and a database 1304. Wired or wireless signals are exchanged between the robotic control system 1302 and autonomous arm 1100 via connection 1301.

In response to control signals received from the robotic control system 1302, the control unit 1115 controls the one or more actuators 1116 to drive the autonomous arm 1100 to move it to an appropriate position for images with an appropriate view to be captured by the imaging device 1102. The control signals are generated by the control processor 1303 in response to signals received from one or more of the arm unit 1114, imaging device 1102 and any other signal sources (not shown). The values of the signals received by the control processor 1303 are compared to signal values stored in the database 1304 along with corresponding arm position information. The control processor 1303 retrieves from the database 1304 arm position information associated with the values of the received signals. The control processor 1303 then generates the control signals to be transmitted to the control unit 1115 using the retrieved arm position information.

For example, if signals received from the imaging device 1102 indicate a predetermined surgical scenario (e.g. via neural network image classification process or the like), the predetermined surgical scenario is looked up in the database 1304 and arm position information associated with the predetermined surgical scenario is retrieved from the database. As another example, if signals indicate a value of resistance measured by the one or more force sensors 1117 of the arm unit 1114, the value of resistance is looked up in the database 1203 and arm position information associated with the value of resistance is retrieved from the database (e.g. to allow the position of the arm to be changed to an alternative position if an increased resistance corresponds to an obstacle in the arm's path). In either case, the control processor 1303 then sends signals to the control unit 1115 to control the one or more actuators 1116 to change the position of the arm to that indicated by the retrieved arm position information.

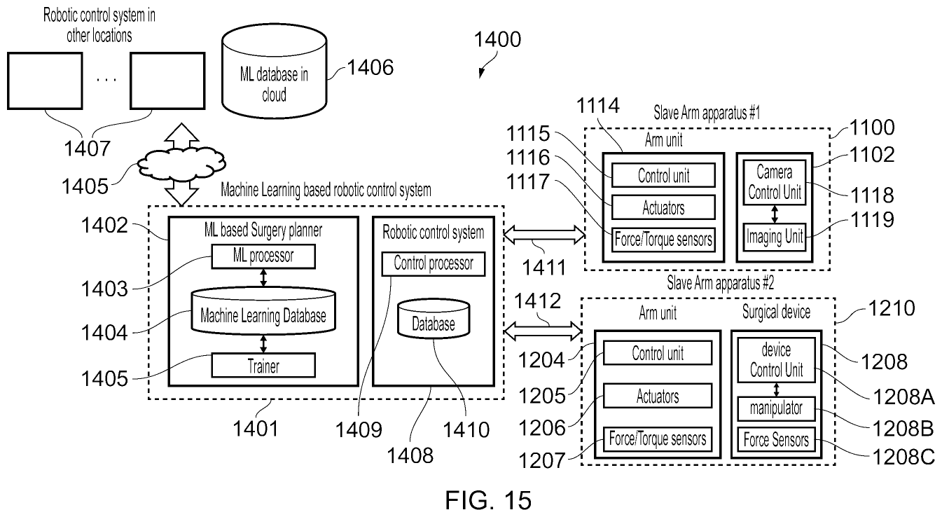

FIG. 15 schematically shows another example of a computer assisted surgery system 1400 to which the present technique is applicable. The system includes one or more autonomous arms 1100 with an imaging unit 1102 and one or more autonomous arms 1210 with a surgical device 1210. The one or more autonomous arms 1100 and one or more autonomous arms 1210 are the same as those previously described. Each of the autonomous arms 1100 and 1210 is controlled by a robotic control system 1408 including a control processor 1409 and database 1410. Wired or wireless signals are transmitted between the robotic control system 1408 and each of the autonomous arms 1100 and 1210 via connections 1411 and 1412, respectively. The robotic control system 1408 performs the functions of the previously described robotic control systems 1111 and/or 1302 for controlling each of the autonomous arms 1100 and performs the functions of the previously described robotic control system 1201 for controlling each of the autonomous arms 1210.

The autonomous arms 1100 and 1210 perform at least a part of the surgery completely autonomously (e.g. when the system 1400 is an open surgery system). The robotic control system 1408 controls the autonomous arms 1100 and 1210 to perform predetermined actions during the surgery based on input information indicative of the current stage of the surgery and/or events happening in the surgery. For example, the input information includes images captured by the image capture device 1102. The input information may also include sounds captured by a microphone (not shown), detection of in-use surgical instruments based on motion sensors comprised with the surgical instruments (not shown) and/or any other suitable input information.

The input information is analysed using a suitable machine learning (ML) algorithm (e.g. a suitable artificial neural network) implemented by machine learning based surgery planning apparatus 1402. The planning apparatus 1402 includes a machine learning processor 1403, a machine learning database 1404 and a trainer 1405.

The machine learning database 1404 includes information indicating classifications of surgical stages (e.g. making an incision, removing an organ or applying stitches) and/or surgical events (e.g. a bleed or a patient parameter falling outside a predetermined range) and input information known in advance to correspond to those classifications (e.g. one or more images captured by the imaging device 1102 during each classified surgical stage and/or surgical event). The machine learning database 1404 is populated during a training phase by providing information indicating each classification and corresponding input information to the trainer 1405. The trainer 1405 then uses this information to train the machine learning algorithm (e.g. by using the information to determine suitable artificial neural network parameters). The machine learning algorithm is implemented by the machine learning processor 1403.

Once trained, previously unseen input information (e.g. newly captured images of a surgical scene) can be classified by the machine learning algorithm to determine a surgical stage and/or surgical event associated with that input information. The machine learning database also includes action information indicating the actions to be undertaken by each of the autonomous arms 1100 and 1210 in response to each surgical stage and/or surgical event stored in the machine learning database (e.g. controlling the autonomous arm 1210 to make the incision at the relevant location for the surgical stage “making an incision” and controlling the autonomous arm 1210 to perform an appropriate cauterisation for the surgical event “bleed”). The machine learning based surgery planner 1402 is therefore able to determine the relevant action to be taken by the autonomous arms 1100 and/or 1210 in response to the surgical stage and/or surgical event classification output by the machine learning algorithm. Information indicating the relevant action is provided to the robotic control system 1408 which, in turn, provides signals to the autonomous arms 1100 and/or 1210 to cause the relevant action to be performed.

The planning apparatus 1402 may be included within a control unit 1401 with the robotic control system 1408, thereby allowing direct electronic communication between the planning apparatus 1402 and robotic control system 1408. Alternatively or in addition, the robotic control system 1408 may receive signals from other devices 1407 over a communications network 1405 (e.g. the internet). This allows the autonomous arms 1100 and 1210 to be remotely controlled based on processing carried out by these other devices 1407. In an example, the devices 1407 are cloud servers with sufficient processing power to quickly implement complex machine learning algorithms, thereby arriving at more reliable surgical stage and/or surgical event classifications. Different machine learning algorithms may be implemented by different respective devices 1407 using the same training data stored in an external (e.g. cloud based) machine learning database 1406 accessible by each of the devices. Each device 1407 therefore does not need its own machine learning database (like machine learning database 1404 of planning apparatus 1402) and the training data can be updated and made available to all devices 1407 centrally. Each of the devices 1407 still includes a trainer (like trainer 1405) and machine learning processor (like machine learning processor 1403) to implement its respective machine learning algorithm.

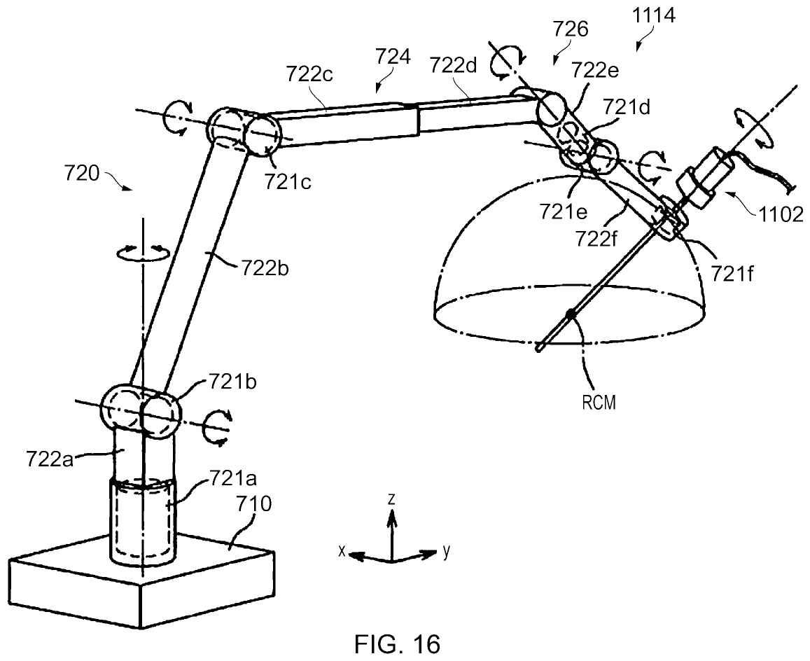

FIG. 16 shows an example of the arm unit 1114. The arm unit 1204 is configured in the same way. In this example, the arm unit 1114 supports an endoscope as an imaging device 1102. However, in another example, a different imaging device 1102 or surgical device 1103 (in the case of arm unit 1114) or 1208 (in the case of arm unit 1204) is supported.

The arm unit 1114 includes a base 710 and an arm 720 extending from the base 720. The arm 720 includes a plurality of active joints 721a to 721f and supports the endoscope 1102 at a distal end of the arm 720. The links 722a to 722f are substantially rod-shaped members. Ends of the plurality of links 722a to 722f are connected to each other by active joints 721a to 721f, a passive slide mechanism 724 and a passive joint 726. The base unit 710 acts as a fulcrum so that an arm shape extends from the base 710.

A position and a posture of the endoscope 1102 are controlled by driving and controlling actuators provided in the active joints 721a to 721f of the arm 720. According to the this example, a distal end of the endoscope 1102 is caused to enter a patient's body cavity, which is a treatment site, and captures an image of the treatment site. However, the endoscope 1102 may instead be another device such as another imaging device or a surgical device. More generally, a device held at the end of the arm 720 is referred to as a distal unit or distal device.

Here, the arm unit 700 is described by defining coordinate axes as follows. Furthermore, a vertical direction, a longitudinal direction, and a horizontal direction are defined according to the coordinate axes. In other words, a vertical direction with respect to the base 710 installed on the floor surface is defined as a z-axis direction and the vertical direction. Furthermore, a direction orthogonal to the z axis, the direction in which the arm 720 is extended from the base 710 (in other words, a direction in which the endoscope 1102 is positioned with respect to the base 710) is defined as a y-axis direction and the longitudinal direction. Moreover, a direction orthogonal to the y-axis and z-axis is defined as an x-axis direction and the horizontal direction.

The active joints 721a to 721f connect the links to each other to be rotatable. The active joints 721a to 721f have the actuators, and have each rotation mechanism that is driven to rotate about a predetermined rotation axis by drive of the actuator. As the rotational drive of each of the active joints 721a to 721f is controlled, it is possible to control the drive of the arm 720, for example, to extend or contract (fold) the arm unit 720.

The passive slide mechanism 724 is an aspect of a passive form change mechanism, and connects the link 722c and the link 722d to each other to be movable forward and rearward along a predetermined direction. The passive slide mechanism 724 is operated to move forward and rearward by, for example, a user, and a distance between the active joint 721c at one end side of the link 722c and the passive joint 726 is variable. With the configuration, the whole form of the arm unit 720 can be changed.

The passive joint 736 is an aspect of the passive form change mechanism, and connects the link 722d and the link 722e to each other to be rotatable. The passive joint 726 is operated to rotate by, for example, the user, and an angle formed between the link 722d and the link 722e is variable. With the configuration, the whole form of the arm unit 720 can be changed.

In an embodiment, the arm unit 1114 has the six active joints 721a to 721f, and six degrees of freedom are realized regarding the drive of the arm 720. That is, the passive slide mechanism 726 and the passive joint 726 are not objects to be subjected to the drive control while the drive control of the arm unit 1114 is realized by the drive control of the six active joints 721a to 721f.

Specifically, the active joints 721a, 721d, and 721f are provided so as to have each long axis direction of the connected links 722a and 722e and a capturing direction of the connected endoscope 1102 as a rotational axis direction. The active joints 721b, 721c, and 721e are provided so as to have the x-axis direction, which is a direction in which a connection angle of each of the connected links 722a to 722c, 722e, and 722f and the endoscope 1102 is changed within a y-z plane (a plane defined by the y axis and the z axis), as a rotation axis direction. In this manner, the active joints 721a, 721d, and 721f have a function of performing so-called yawing, and the active joints 421b, 421c, and 421e have a function of performing so-called pitching.

Since the six degrees of freedom are realized with respect to the drive of the arm 720 in the arm unit 1114, the endoscope 1102 can be freely moved within a movable range of the arm 720. A hemisphere as an example of the movable range of the endoscope 723. Assuming that a central point RCM (remote center of motion) of the hemisphere is a capturing centre of a treatment site captured by the endoscope 1102, it is possible to capture the treatment site from various angles by moving the endoscope 1102 on a spherical surface of the hemisphere in a state where the capturing centre of the endoscope 1102 is fixed at the centre point of the hemisphere.

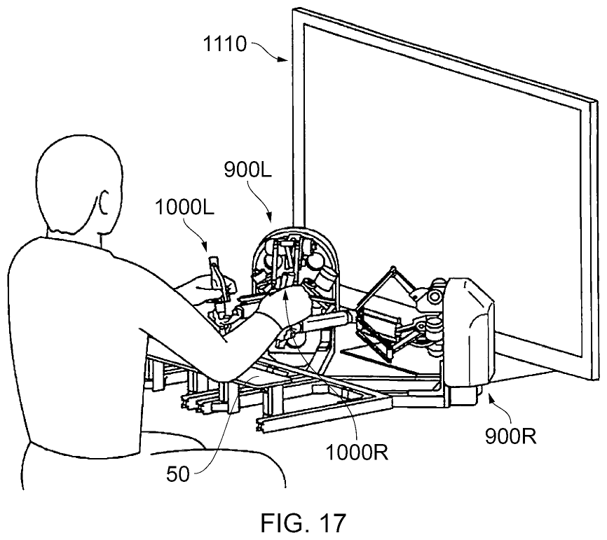

FIG. 17 shows an example of the master console 1104. Two control portions 900R and 900L for a right hand and a left hand are provided. A surgeon puts both arms or both elbows on the supporting base 50, and uses the right hand and the left hand to grasp the operation portions 1000R and 1000L, respectively. In this state, the surgeon operates the operation portions 1000R and 1000L while watching electronic display 1110 showing a surgical site. The surgeon may displace the positions or directions of the respective operation portions 1000R and 1000L to remotely operate the positions or directions of surgical instruments attached to one or more slave apparatuses or use each surgical instrument to perform a grasping operation.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

Embodiments of the present technique can generally described by the following numbered clauses:

(1)
A device, comprising circuitry configured to: receive surgical data indicating a parameter of a surgical instrument used during a surgical procedure and a stress related parameter of a member of a surgical team performing the surgical procedure; and associating the surgical data with the stress related parameter.

(2)
The device according to clause 1, wherein the surgical procedure is formed of a plurality of subprocedures, and the circuitry is configured to divide the surgical procedure into a plurality of sections, each section comprising the surgical data and the stress related parameter relating to a different subprocedure.

(3)
The device according to either one of clause 1 or 2, wherein circuitry is configured to receive image data of the surgical procedure and the image data of the surgical procedure is image data obtained by the surgical instrument.

(4)
The device according to any preceding clause, wherein the surgical data includes movement data of a robotic surgical instrument.

(5)
The device according to any preceding clause, wherein the circuitry is configured to receive the stress related parameter from a wearable device.

(6)
The device according to any preceding clause, wherein the member of the surgical team is a surgeon.

(7)
A surgical training apparatus comprising circuitry configured to: receive the surgical data and the associated stress related parameter from the device of any one of clauses 1 to 6; and
generate a surgical training simulation based on the surgical data and the stress related parameter.

(8)
The surgical training apparatus according to clause 7 wherein the circuitry is further configured to: receive a second stress related parameter from the member of the surgical team using the surgical training apparatus; and generate the surgical training simulation based on the second stress related parameter.

(9)
The surgical training apparatus according to clause 8, wherein the circuitry is further configured to: generate the surgical training simulation based on a value of the stress related parameter that is higher than the second stress related parameter.

(10)
The surgical training apparatus according to any one of clauses 7, 8 and 9, wherein the circuitry is configured to: control a display to display the generated surgical training simulation.

(11)
The surgical training apparatus according to clause 10, wherein the circuitry is configured to control the display to display a training graph defining the surgical training simulation.

(12)
A surgical training system comprising a device according to any one of claims 1 to 6 connected to a surgical training apparatus according to any one of clauses 7 to 11.

(13)
A method comprising: receiving surgical data indicating a parameter of a surgical instrument used during a surgical procedure and a stress related parameter of a member of a surgical team performing the surgical procedure; and associating the surgical data with the stress related parameter.

(14)
The method according to clause 13, wherein the surgical procedure is formed of a plurality of subprocedures, and the method comprises: dividing the surgical procedure into a plurality of sections, each section comprising the surgical data and the stress related parameter relating to a different subprocedure.

(15)
The method according to clause 13 or 14, comprising: receiving image data of the surgical procedure and the image data of the surgical procedure is image data obtained by the surgical instrument.

(16)
The method according to any one of clauses 13 to 15, wherein the surgical data includes movement data of a robotic surgical instrument.

(17)
The method according to any one of clauses 13 to 16, comprising: receiving the stress related parameter from a wearable device.

(18)
The method according to any one of clauses 13 to 17, wherein the member of the surgical team is a surgeon.

(19)
A surgical training method comprising: receiving the surgical data and the associated stress related parameter from the device of any one of clauses 13 to 18; and generating a surgical training simulation based on the surgical data and the stress related parameter.

(20)
The surgical training method according to clause 19 comprising: receiving a second stress related parameter from the member of the surgical team using the surgical training apparatus; and generating the surgical training simulation based on the second stress related parameter.

(21)
The surgical training method according to clause 20, wherein the circuitry is further configured to: generate the surgical training simulation based on a value of the stress related parameter that is higher than the second stress related parameter.

(22)
The surgical training method according to any one of clauses 19, 20 and 21, comprising: controlling a display to display the generated surgical training simulation.

(23)
The surgical training method according to clause 22, comprising: controlling the display to display a training graph defining the surgical training simulation.

(24)
A computer program product comprising computer readable instructions which, when loaded onto a computer, configures the computer to perform a method according to any one of clauses 13 to 23.

### REFERENCES

NPL 1: “Effects of stress on heart rate complexity—A comparison between short-term and chronic stress”; C. Schubert, M. Lambatz, R. A. Nelesen, W. Bardwell, J-B. Choi and J. E. Dimsdale, Bio Psychol. 2009 March; 80(3): 325-332 (see https://www.ncbi.nlm.nih.gov/pmc/articles/PMC2653595/)

1. A device, comprising circuitry configured to: receive surgical data indicating a parameter of a surgical instrument used during a surgical procedure and a stress related parameter of a member of a surgical team performing the surgical procedure; and associating the surgical data with the stress related parameter.

2. The device according to claim 1, wherein the surgical procedure is formed of a plurality of subprocedures, and the circuitry is configured to divide the surgical procedure into a plurality of sections, each section comprising the surgical data and the stress related parameter relating to a different subprocedure.

3. The device according claim 1, wherein circuitry is configured to receive image data of the surgical procedure and the image data of the surgical procedure is image data obtained by the surgical instrument.

4. The device according to claim 1, wherein the surgical data includes movement data of a robotic surgical instrument.

5. The device according to claim 1, wherein the circuitry is configured to receive the stress related parameter from a wearable device.

6. The device according to a claim 1, wherein the member of the surgical team is a surgeon.

7. A surgical training apparatus comprising circuitry configured to: receive the surgical data and the associated stress related parameter from the device of claim 1; and
generate a surgical training simulation based on the surgical data and the stress related parameter.

8. The surgical training apparatus according to claim 7 wherein the circuitry is further configured to: receive a second stress related parameter from the member of the surgical team using the surgical training apparatus; and generate the surgical training simulation based on the second stress related parameter.

9. The surgical training apparatus according to claim 8, wherein the circuitry is further configured to: generate the surgical training simulation based on a value of the stress related parameter that is higher than the second stress related parameter.

10. The surgical training apparatus according to claim 7, wherein the circuitry is configured to: control a display to display the generated surgical training simulation.

11. The surgical training apparatus according to claim 10, wherein the circuitry is configured to control the display to display a training graph defining the surgical training simulation.

12. A surgical training system comprising a device according to claim 1 connected to a surgical training apparatus that is configured to generate a surgical training simulation based on the surgical data and the stress related parameter.

13. A method comprising: receiving surgical data indicating a parameter of a surgical instrument used during a surgical procedure and a stress related parameter of a member of a surgical team performing the surgical procedure; and associating the surgical data with the stress related parameter.

14. The method according to claim 13, wherein the surgical procedure is formed of a plurality of subprocedures, and the method comprises: dividing the surgical procedure into a plurality of sections, each section comprising the surgical data and the stress related parameter relating to a different subprocedure.

15. The method according to claim 13, comprising: receiving image data of the surgical procedure and the image data of the surgical procedure is image data obtained by the surgical instrument.

16. The method according to claim 13, wherein the surgical data includes movement data of a robotic surgical instrument.

17. The method according to claim 13, comprising: receiving the stress related parameter from a wearable device.

18. The method according to claim 13, wherein the member of the surgical team is a surgeon.

19. A surgical training method comprising: receiving the surgical data and the associated stress related parameter from the device of claim 13; and
generating a surgical training simulation based on the surgical data and the stress related parameter.

20. The surgical training method according to claim 19 comprising: receiving a second stress related parameter from the member of the surgical team using the surgical training apparatus; and generating the surgical training simulation based on the second stress related parameter.

21. The surgical training method according to claim 20, wherein the circuitry is further configured to: generate the surgical training simulation based on a value of the stress related parameter that is higher than the second stress related parameter.

22. The surgical training method according to claim 19, comprising: controlling a display to display the generated surgical training simulation.

23. The surgical training method according to claim 22, comprising: controlling the display to display a training graph defining the surgical training simulation.

24. A computer program product comprising computer readable instructions which, when loaded onto a computer, configures the computer to perform a method according to claim 13.
